(19) United States
(12) Patent Application Publication
(10) Pub. No.: US 2020/0242566 A1
(43) Pub. Date: Jul. 30, 2020

(54) SYSTEM AND METHOD OF PRIORITIZING PATIENTS FOR CLINICIAN MANAGEMENT

(71) Applicant: PACESETTER, INC., Sylmar, CA (US)

(72) Inventors: Rahul Agarwal, Los Angeles, CA (US); Rupinder Bharmi, Canyon Country, CA (US); Matthew S.D. Kerr, Valencia, CA (US); Phillip B. Adamson, Austin, TX (US); Nirav Dalal, Porter Ranch, CA (US)

(21) Appl. No.: 16/262,592

(22) Filed: Jan. 30, 2019

(51) Int. Cl. G06Q 10/10; A61B 5/00; A61B 5/021; G06N 7/00; G16H 50/20

(52) U.S. Cl. CPC G06Q 10/1095; A61B 5/7275; A61B 5/021; A61B 5/024; G06N 7/005; G16H 50/20; A61B 5/4848

(57) ABSTRACT

A system for prioritizing patients for clinician management that includes one or more processors configured to execute program instructions. When executed the one or more processors determine a risk score for a patient, alter the risk score based on patient based parameters to determine a priority score of the patient, compare the priority score of the patient to priority scores of other patients, assign a rank to the patient based on the comparison of the priority score of the patient to the priority scores of the other patients, and schedule an appointment for the patient based on the rank.

### BACKGROUND

Embodiments of the present disclosure generally relate to a system and method of prioritizing patients for clinician management.

Heart failure (HF)-related hospitalizations costs per congestive heart failure (CHF) patient are a major health concern in the United States. Patients are usually admitted to the hospital for HF because of worsening signs and symptoms of CHF. Increases in intra-cardiac and pulmonary artery pressures have been shown as a cause of this CHF.

Implantable sensors are utilized to passively measure the pulmonary artery pressure (PAP), providing a direct measurement of cardiac filling pressures that guide individual medical therapy intended to maintain long-term stability, thus preventing decompensation requiring hospitalization.

However, such clinical management requires at least weekly manual review of PAP by physicians and nurses. For high volume clinics this limits scalability of measuring PAP of every patient on a weekly basis as a result of personnel requirements. Therefore, a need remains to scale hemodynamics-guided heart failure management to accommodate high volume patient intake.

### BRIEF SUMMARY

In accordance with embodiments herein, a system is provided for prioritizing patients for clinician management that includes one or more processors configured to execute program instructions. When executed the one or more processors determine a risk score for a patient, alter the risk score based on patient based parameters to determine a priority score of the patient, and compare the priority score of the patient to priority scores of other patients. When the instructions are executed, the one or more processors also assign a rank to the patient based on the comparison of the priority score of the patient to the priority scores of the other patients, and schedule an appointment for the patient based on the rank.

Optionally, the determining the priority score for a patient includes estimating a probability of a predetermined event to determine the risk score for the patient, and determining a time period since a last appointment. The determining the priority score for a patient also includes receiving medication titration data, and inputting the probability of the predetermined event, the time period since the last appointment, and medication titration data into a patient prioritization algorithm. Optionally, estimating the probability of the predetermined event includes monitoring pulmonary artery pressure with a sensor to detect pulmonary artery pressure data, recording the pulmonary artery pressure data detected by the sensor, and calculating a risk estimate based on the recorded pulmonary artery pressure data.

In one aspect, calculating the risk estimate includes determining one of patient heart rate or medication usage. Optionally, the probability of the predetermined event also includes calculating the probability of the predetermined event based on historical data. Alternatively, the historical data includes one of previous patient risk estimates, patient co-morbidities, demographics, or medication changes.

In one example, the predetermined event is heart failure.

Optionally, determining the risk score for the patient also includes forming a linear model, receiving systolic pulmonary artery pressure, diastolic pulmonary artery pressure, and heart rate data from an existing group of patients, and creating a generalizable estimator based on the received systolic pulmonary artery pressure, diastolic pulmonary artery pressure, and heart rate data from an existing group of patients. Determining the risk score for the patient can also include monitoring pulmonary artery pressure with a sensor to detect pulmonary artery pressure data, and utilizing the pulmonary artery pressure in with the generalizable estimator.

In one aspect, determining the risk score for the patient also includes forming a non-linear model, monitoring pulmonary artery pressure with a sensor to detect pulmonary artery pressure data, utilizing the pulmonary artery pressure data in the non-linear model.

In accordance with other embodiments herein, a system for prioritizing patients for clinician management is provided that includes one or more processors configured to execute program instructions. When program instructions are executed, the one or more processors receive a risk score for a patient inputted into the one or more processors at an interface, and determine a first variable related to the patient based on historical data. When program instructions are executed, the one or more processors also alter the risk score by a predetermined amount based on the determined first variable to provide an updated risk score, determine a second variable related to the patient based on sensor data received by the one or more processors, and alter the updated risk score based on the determined second variable to provide a priority score of the patient. When program instructions are executed, the one or more processors also compare the priority score of the patient to priority scores of other patients, assign a ranking to the patient based on the comparison of the priority score of the patient to the priority scores of the other patients, and schedule an appointment for the patient based on the ranking.

Optionally, the historical data includes one of previous patient risk estimates, patient co-morbidities, demographics, or medication changes. In one aspect altering the risk score by a predetermined amount includes subtracting from the risk score.

In accordance with embodiments herein a method of prioritizing patients for clinician management is provided that includes monitoring pulmonary artery pressure with a sensor to detect pulmonary artery pressure data, and estimating the probability a patient will have a heart failure event during a predetermined period based on the detected pulmonary artery pressure data. The method also includes determining a risk score for the patient based on the probability the patient will have the heart failure event during a predetermined period, altering the risk score based on patient based parameters to determine a priority score of the patient, and comparing the priority score of the patient to priority scores of other patients. The method also includes assigning a rank to the patient based on the comparison of the priority score of the patient to the priority scores of the other patients, and scheduling an appointment for the patient based on the rank.

Optionally, determining the priority score for the patient includes determining a time period since a last appointment, receiving medication titration data, and inputting the probability the patient will have the heart failure event during the predetermined period, the time period since the last appointment, and medication titration data into a patient prioritization algorithm.

In one aspect, the method also includes determining a first weight related to the inputted probability the patient will have the heart failure event during the predetermined period, determining a second weight related to the Inputted time period since the last appointment, and determining a third weight related to the inputted medication titration data.

In one example the medication titration data includes change in medication usage data

Optionally, estimating the probability the patient will have the heart failure event during the predetermined period based on the detected pulmonary artery pressure data also includes receiving historical data related to the probability the patient will have the heart failure event during the predetermined period, and comparing the historical data to the detected pulmonary artery pressure data.

In one aspect, estimating the probability the patient will have the heart failure event during the predetermined period based on the detected pulmonary artery pressure data also includes receiving historical data related to the probability the patient will have the heart failure event during the predetermined period, and inputting the historical data into a risk score algorithm.

Optionally, the historical data includes one of previous patient risk estimates, patient co-morbidities, demographics, or medication changes.

In one aspect, detecting pulmonary artery pressure data includes forming a pulmonary pressure waveform and extracting the detected pulmonary artery pressure data from the detected pulmonary pressure waveform, and the risk score is determined utilizing the risk score algorithm and the detected pulmonary artery pressure data.

### BRIEF DESCRIPTION OF THE DRAWINGS

FIG. 1 illustrates a schematic diagram of clinician management system operated in accordance with embodiments herein.

FIG. 2 illustrates a block flow diagram of a method of prioritizing patients for clinician management in accordance with embodiments herein.

FIG. 3 illustrates a schematic block diagram of a method to determine patient-specific priority rank in accordance with embodiments herein.

FIG. 4 illustrates a schematic block diagram of a method to determine patient-specific priority rank in accordance with embodiments herein.

FIG. 5 illustrates a schematic block diagram of a method to determine patient-specific priority rank in accordance with embodiments herein.

FIG. 6 illustrates a schematic display of a clinician management system in accordance with embodiments herein.

FIG. 7 illustrates a schematic display of a clinician management system in accordance with embodiments herein.

FIG. 8 illustrates a schematic block diagram of a distributed processing system in accordance with embodiments herein.

### DETAILED DESCRIPTION

It will be readily understood that the components of the embodiments as generally described and illustrated in the figures herein, may be arranged and designed in a wide variety of different configurations in addition to the described example embodiments. Thus, the following more detailed description of the example embodiments, as represented in the figures, is not intended to limit the scope of the embodiments, as claimed, but is merely representative of example embodiments.

Reference throughout this specification to “one embodiment” or “an embodiment” (or the like) means that a particular feature, structure, or characteristic described in connection with the embodiment is included in at least one embodiment. Thus, appearances of the phrases “in one embodiment” or “in an embodiment” or the like in various places throughout this specification are not necessarily all referring to the same embodiment.

The term “obtain” or “obtaining”, as used in connection with data, signals, information and the like, includes at least one of i) accessing memory of an external device or remote server where the data, signals, information, etc. are stored, ii) receiving the data, signals, information, etc. over a wireless communications link between a medical device, such as an implantable medical device (IMD), and a local external device, and/or iii) receiving the data, signals, information, etc. at a remote server over a network connection. The obtaining operation, when from the perspective of an IMD, may include sensing new signals in real time, and/or accessing memory to read stored data, signals, information, etc. from memory within the IMD. The obtaining operation, when from the perspective of a local external device, includes receiving the data, signals, information, etc. at a transceiver of the local external device where the data, signals, information, etc, are transmitted from an IMD and/or a remote server. The obtaining operation may be from the perspective of a remote server, such as when receiving the data, signals, information, etc. at a network interface from a local external device and/or directly from an IMD. The remote server may also obtain the data, signals, information, etc. from local memory and/or from other memory, such as within a cloud storage environment and/or from the memory of a workstation or clinician external programmer.

Embodiments may be implemented in connection with one or more IMDs. Non-limiting examples of IMDs include one or more of neurostimulator devices, implantable leadless monitoring and/or therapy devices, and/or alternative implantable medical devices. For example, the IMD may represent a cardiac monitoring device, pacemaker, cardioverter, cardiac rhythm management device, defibrillator, neurostimulator, leadless monitoring device, leadless pacemaker and the like. For example, the IMD may include one or more structural and/or functional aspects of the device(s) described in U.S. Pat. No. 9,333,351 “Neurostimulation Method And System To Treat Apnea” and U.S. Pat. No. 9,044,610 “System And Methods For Providing A Distributed Virtual Stimulation Cathode For Use With An Implantable Neurostimulation System”, which are hereby incorporated by reference. Additionally or alternatively, the IMD may include one or more structural and/or functional aspects of the device(s) described in U.S. Pat. No. 9,216,285 “Leadless Implantable Medical Device Having Removable And Fixed Components” and U.S. Pat. No. 8,831,747 “Leadless Neurostimulation Device And Method including The Same”, which are hereby incorporated by reference. Additionally or alternatively, the IMD may include one or more structural and/or functional aspects of the device(s) described in U.S. Pat. No. 8,391,980 “Method And System For Identifying A Potential Lead Failure In An Implantable Medical Device” and U.S. Pat. No. 9,232,485 “System And Method For Selectively Communicating With An Implantable Medical Device”, which are hereby incorporated by reference.

Additionally or alternatively, the IMD may be a subcutaneous IMD that includes one or more structural and/or functional aspects of the device(s) described in U.S. application Ser. No.: 15/973,195, titled “Subcutaneous Implantation Medical Device With Multiple Parasternal-Anterior Electrodes” and filed May 7, 2018; U.S. application Ser. No.: 15/973,219, titled “Implantable Medical Systems And Methods Including Pulse Generators And Leads” filed May 7, 2018; U.S. application Ser. No.: 15/973,249, titled “Single Site Implantation Methods For Medical Devices Having Multiple Leads”, filed May 7, 2018, which are hereby incorporated by reference in their entireties. Further, one or more combinations of IMDs may be utilized from the above incorporated patents and applications in accordance with embodiments herein.

Embodiments may be implemented in connection with one or more sensor, monitor, and/or device that measures pulmonary artery pressure. Such sensors, monitors, and/or devices may include one or more structural and/or functional aspects of the device(s) described in U.S. Pat. No. 8,665,086, titled “Physiological Data Acquisition and Management System for Use with an Implanted Wireless Sensor” filed Jan. 4, 2012; U.S. Pat. No. 8,264,240, titled “Physical Property Sensor with Active Electronic Circuit and Wireless Power and Data Transmission” filed Jul. 20, 2009; U.S. Pat. No. 8,025,625, titled “Sensor with Electromagnetically Coupled Hermetic Pressure Reference” filed Apr. 12, 2006; U.S. Pat. No. 7,966,886, titled “Method and Apparatus for Measuring Pressure Inside a Fluid System” filed Oct. 9, 2009; and U.S. Pat. No. 7,909,770, titled “Method for Using a Wireless Pressure Sensor to Monitor Pressure Inside the Human Heart: filed Jul. 3, 2007, which are hereby incorporated by reference in their entireties.

Embodiments herein provide methods and systems for prioritizing patients for clinician management using one or more processors. In embodiments patients are provided with a risk score related to the probability or likelihood the patient will have a heart failure (HF) that will require hospitalization within a given period. In one example the given period is one week. The risk score may be determined through an algorithm, linear modeling, non-linear modeling, and the like based on patient data, patient group data, historical data, and the like. The risk score and accompanying data may then be used by a clinician in diagnosis and treatment of the patient in an efficient manner. As such, medication changes may be done remotely, instead of at an at the office visit.

When an in office appointment, or visit is required, an algorithm is then utilized based on the risk score and patient specific parameters to calculate a priority score. The patient based parameters can include time since a last PAP measurement, medication usage, changes in risk score, changes in medication prescription, and the like. Once the priority score of a patient is determined, the priority score of the patient is compared to other patient risk scores and the patient is prioritized accordingly.

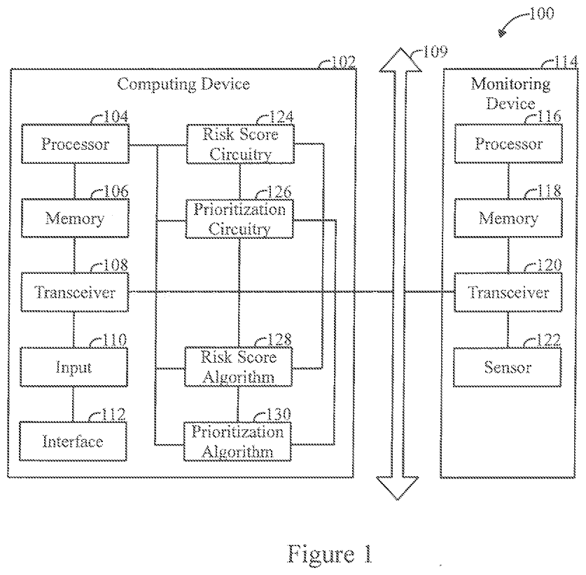

FIG. 1 illustrates a schematic diagram of clinician management system 100. The clinician management system 100 includes one or more computing devices 102 that includes one or more processors 104, a memory 106, transceiver 108 for corresponding over a communication link 109, an input 110, and an interface 112. The clinician management system 100 also includes a monitoring device 114 that includes one or more processors 116, a memory 118, a transceiver 120 for communicating over the communication link 109, and a sensor 122.

The sensor 122 of the monitoring device 114 may be a single sensor or a plurality of sensors that communicate either with one another and/or the one or more processors 116 of the monitoring device 114. The sensor 122 in one example is an implantable sensor that passively measures the pulmonary artery pressure (PAP) of a patient. In particular, the sensor alternatively could another heart monitoring sensor. In one example the sensor detects PAP and transmits pulmonary artery pressure data to the one or more processors 104.

The clinician management system 100 also includes risk score circuitry 124, and prioritization circuitry 126. In one example, the risk score circuitry 124 and prioritization circuitry 126 are within the one or more computing devices 102. Additionally or alternatively, the risk score circuitry 124 and prioritization circuitry 126 are in the monitoring device 114 or other remote device to the one or more computing devices 102. In another embodiment, risk score circuitry 124 and prioritization circuitry 126 are not provided and determination related to risk score and prioritization are made by software of the one or more processors 104 of the one or more computing devices 102, the one or more processors 116 of the monitoring device 114, in the cloud, and the like.

In one example a risk score algorithm 128 is utilized by the clinician management system 100 to determine the risk score and the priority score is determined by a prioritization algorithm 130. In one example the prioritization circuitry 126 controls and operates the prioritization algorithm 130, while alternatively, the prioritization algorithm is within software, or in communication with, one or more processors 104 of the clinician management system 100. The prioritization algorithm 130 in one example utilizes reinforcement learning and is considered an artificial intelligence (AI) or machine learning type algorithm. In particular, the prioritization algorithm 130 is utilized to determine a patient prioritization ranking based on an initial patient risk score that can be determined by the risk score algorithm 128 to schedule future patient appointments.

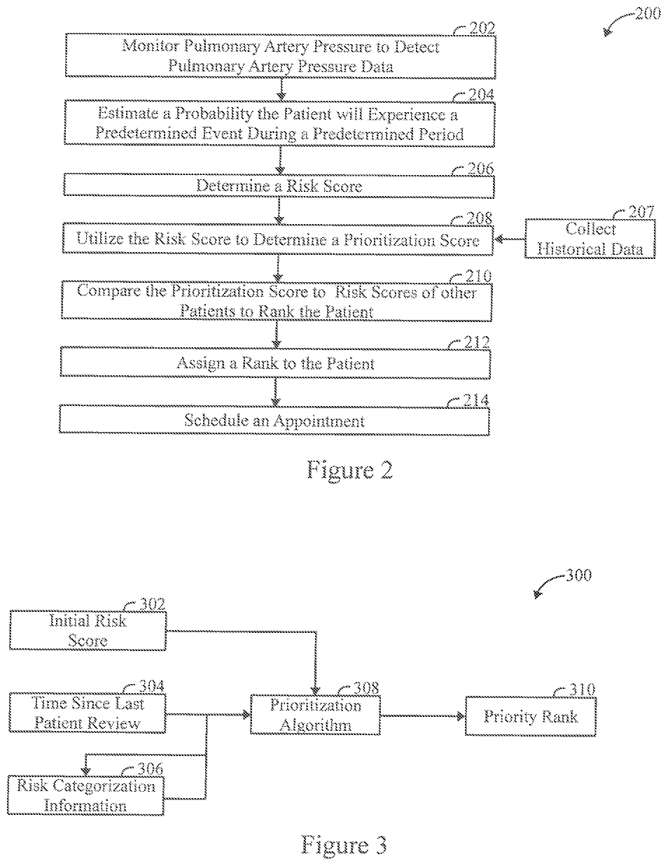

FIG. 2 illustrates a method 200 of prioritizing patients for clinician management. In one example the clinician management system 100 of FIG. 1 is utilized to perform the method 200 provided. The method may be performed through computer implemented hardware, software, circuitry, and the like. Optionally, the method 200 is performed in association with, or within a heart failure clinic. Alternatively, the method 200 is performed in association with a general practice facility, an emergency care facility, a hospital, a pediatric clinic, a nursing home or care facility with in-patient care, and the like. Information and/or data monitored, detected, inputted, received, shared, communicated, transmitted, and the like may be obtained by local equipment, local computing devices, and the like, or by remote equipment, computing devices, the cloud, and the like, Specifically, hospitals, heart failure clinics, and other such care providers may share and transmit the information and data utilized and such actions may be performed in more than one location and/or by one or more devices or systems.

At 202, pulmonary artery pressure (PAP) is monitored to detect pulmonary artery pressure data. In one example a sensor detects the PAP and this PAP data is communicated to one or more processors 104 of the clinician management system 100 of FIG. 1. In one example the sensor is in communication with a monitoring system that is remote to the one or more processors of the clinician management system and is in communication with the one or more processors with the PAP data transmitted to the clinician management system. Transmission includes wireless and wire based transmissions. Alternatively, the monitoring system is within and part of the clinician management system and PAP data is directly communicated to the one or more processors.

At 204, one or more processors estimate a probability the patient will experience a predetermined event during a predetermined period based on the detected PAP. In one example, the probability is estimated by the one or more processors utilizing a risk score algorithm. Alternatively, the probability is estimated by assigning numerical values to different variables and adding the numeric values together to arrive at a risk score. Variables may include age, detected PAP, preexisting health conditions including kidney disease, heart disease, cancer, high blood pressure, previous heart attack, medication titration, reaction to medication changes, changes in variables including risk score, and the like.

In one example estimating the probability the patient will experience a predetermined event includes determining the time since a last appointment, receiving medication titration data, and inputting the probability the patient will have the heart failure event during a predetermined period, the time since the last appointment, and medication titration data into a prioritization algorithm. Additionally and alternatively, the medication titration data can include the date when medication was altered and the change in medical usage, or dosage, or type of medication.

In another example estimating the probability the patient will experience a predetermined event during a predetermined period includes receiving historical data related to the probability the patient will experience the predetermined event during the predetermined period, and inputting the historical data into an algorithm. Optionally, the historical data, such as previous PAP data of the patient, or a group of patients, is compared to the detected PAP data. Additionally and alternatively, the historical data includes one or previous patient risk estimates, patient co-morbidities, demographics, or medication changes. By using different data and information in estimating the probability the patient will experience a predetermined event, an enhanced evaluation is provided, increasing accuracy and improving patient care. Thus, lives may be prolonged, and costs reduced.

At 206, a risk score for a patient is determined based on the probability the patient will experience the predetermined event during the predetermined period. In one example the predetermined event is heart failure. In one example the predetermined period, or interval, can be one week, two weeks, a month, or the like. As used herein, predetermined period and predetermined interval may be used interchangeably. When used herein, the phrase “the probability the patient will” includes not only a determined percentage or numerical value, but also non-numerical values and corresponding indicators. For example, a risk score algorithm may be used to determine a risk range or risk category a patient falls into with the range or risk category illustrated by color, bars, and the like. Such probability may also include morphological comparisons such as comparisons of waveforms in graphs associated with heart related data. In this manner, probability is simply the factoring or use of event related variable(s) to determine the likelihood an event will or won't occur. Optionally, the event is heart failure during a predetermined period or interval. The probability thus may be represented in numerous manners, including a risk score, a subsequent risk score, a percentage, a bar graph, a range, a color-coded indicator, text indicia providing an indicator text such as “low” “medium”, and “high^{”}, pictorially, and the like.

At 207, the one or more processors collect historical data related to the patient. This includes age, height, weight, medications, changes in medications, previous medical procedures, diagnosed medical conditions, medical history, time since last appointment, family medical history, smoking history, drinking history, drug use history, prior health readings such as blood pressure, PAP data EKG readings, test results, or the like. The one or more processor may receive such information and data from a local memory, a remote processor, a remote memory, the cloud, or from more than one of the local memory, remote processor, remote memory, the cloud, or the like.

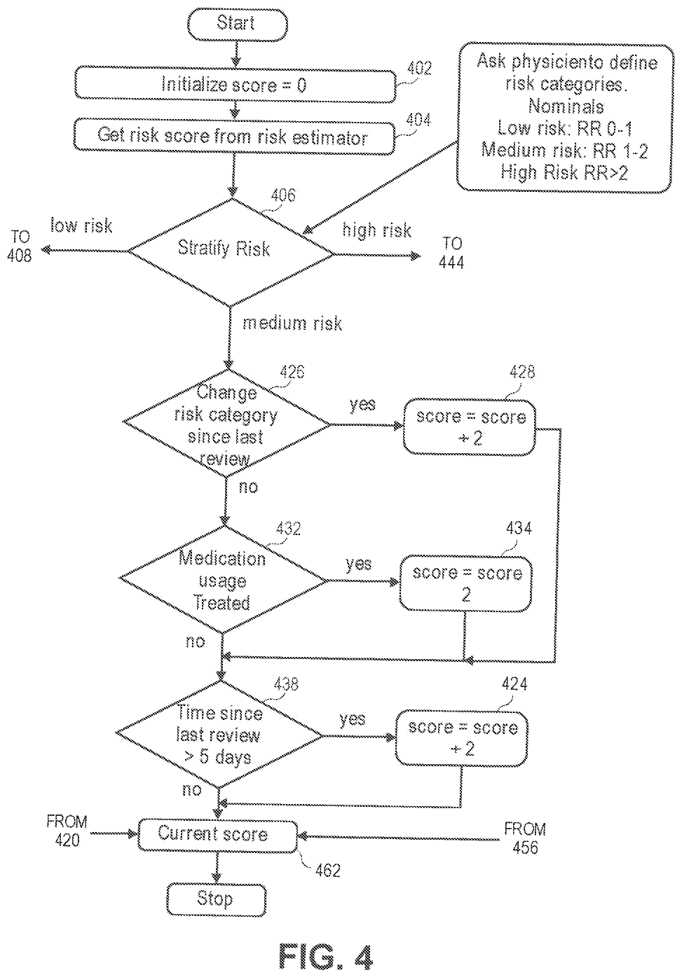

At 208, the one or more processors alters the risk score based on patient based parameters to determine a priority score. In one example, patient parameters include time since a last appointment, visit, or measurement, previous patient risk scores, changes in medication usage, and the like. Patient based parameters may also include data related to a group of patients, such as demographics, percentage of patients with similar test results that experience the event in the predetermined period, effect of medication on similarly situated patients, morphological data, including waveform data of similarly situated patients, and the like. Similarly situated includes patients that have at least one variable in common with a current patient, including approximate age, PAP, medication, and the like. In examples, the patient prioritization algorithms of FIGS. 3-5 are utilized to alter a risk score based on patient parameters to arrive at the priority score for an individual patient. Optionally, multiple estimations, calculations, determinations, operations, or algorithms are utilized to after the risk score and arrive at the priority score. In one example, determining the final patient risk score includes calculating the time since a last appointment, and receiving medication titration data. The probability the patient will have the heart failure event during a predetermined period, the time since the last appointment, and medication titration data into a prioritization algorithm are then inputted into the one or more processors to determine the final patient risk score by using the prioritization algorithm. The medication titration data can include the date when medication was altered and the change in medical usage or dosage or type of medication. The prioritization algorithm in one example provides predetermined weight to each of the risk score, time since last appointment, and medication titration data in determining the priority score. In an example the prioritization algorithm is a machine learning algorithm and modifies the weights over time based on patient data related to previous patients that experience the event, such as a heart failure event of a patient compared to their risk score at the time of the event.

At 210, one or more processors compare the priority score to priority scores of other patients in order to rank the patient compared to other patients. In one example a comparator is utilized to make the comparison. In another example the priority score of all patients is placed on a list that is in numerical order, and the priority score of the patient inserted in order onto the list. Optionally, the list to visually illustrated on an interface with the priority score of the patient next to the list. In this manner, a clinician or scheduler with knowledge of all of the patients may use the information to schedule the patient based on additional information that cannot be quantified by the clinician management system. Such additional information includes known other appointments, ability of individual patients to get to the facilities for tests on certain days, patient specific work schedules, and the like.

At 212, one or more processors assign a rank to the patient based on the comparison of the priority score to the priority scores of the other patients. In an example the rank of the patient is assigned based on an ascending or descending order of a patient on a prioritization list and patient is provided a rank according. Specifically, the patient with the highest score is given a rank of one (1), the patient with the second highest score is given a rank of two (2) and so on. Additionally, in an example where the amount of time since a previous appointment, visit, or measurement is a variable, each patient on the list has their priority score recalculated after a predetermined interval. In one example, the clinician management system automatically updates a patient's score after two days, five days, seven days, ten days, and fourteen days. Optionally, the higher the priority score, the more iterations are provided for updates, thus for a high risk category patient, their score may he updated daily. Alternatively, every patient on the list is updated periodically, such that updates occur independent of when a patient was placed on the list. Thus, the entire list may update daily, weekly, bi-weekly, and the like.

At 214, one or more processors schedules an appointment for the patient based on the patient rank. In one example, based on a category a patient is placed based on theft priority score determines the starting point for when to schedule an appointment. For example, for an individual that is in the low risk category, the one or more processors begin searching for open appointments that are three weeks away and place the patient in the first available time slot more than three weeks away. Alternatively, for a high risk patient, the one or more processors search for open appointments that are two days away and place the patient in the first available time slot more than two days away. Additionally and alternatively, a high risk patient may be given priority, such that a high risk patient must be scheduled within a predetermined period, such as two weeks. In a case when no appointments are available during the two weeks, the one or more processors searches for the patient with the lowest risk score scheduled over the next two weeks, and as long as that patient is not a high risk patient, the one or more processors send a message, through email, text, and the like, and reschedule the scheduled patient to a later time to ensure the high risk patient has priority. If all of the patients over the two week period are high risk, the one or more processors in one example communicate with remote clinician management systems to find a different location where the patient may be scheduled for an appointment, visit, or measurement over the predetermined two week time period.

FIG. 3 illustrates a block diagram of an example method 300 utilized by a clinician management system to determine patient-specific priority rank. In one example the clinician management system is the clinician management system 100 of FIG. 1. In one example this method 300 is performed by the risk score circuitry 124 and prioritization circuitry 126 of FIG. 1. In another example the method 300 is performed by software, including of one or more processors 104 of the one or more computing devices 102, the one or more processors 116 of the monitoring device 114, in the cloud, and the like. In one example, the method 300 is implemented through a patient prioritization algorithm with each determination made through a function or separate algorithm that provides variables for use by the prioritization algorithm.

At 302, one or more processors determine a risk score. In one example the one or more processors utilize a risk score algorithm to determine the risk score. In an example, the risk score algorithm determines a probability of a predetermined event and assigns a risk score according. In one example the predetermined event is a heart failure (HF) within a predetermined interval of time. The predetermined interval may be one day, one week, two weeks, one month, and the like. In one example, numerous variables are utilized by the risk score algorithm in determining the probability a patient will experience heart failure within the predetermined interval of time. The variables include historical data, pulmonary artery pressure, heart rate, medication usage, and the like. The historical data can include previous patient risk estimates, patient co-morbidities, demographics, medication changes, and the like. Based on these variables the risk score algorithm determines the probability of heart failure within the predetermined interval of time in order to determine the risk score.

In another example, the risk score is determined by the one or more processors by forming a linear model. In an example, the linear model is formed by receiving systolic PAP, diastolic PAP, and heart rate data from an existing group of patients and creating a generalizable estimator based on the received systolic PAP, diastolic PAP, and heart rate data. In one example the generalizable estimator utilizes measured pulmonary artery pressure to form the linear model. In yet another example, the risk score is determined by forming a non-linear model. In one example, PAP data, systolic PAP, diastolic PAP, historical data, and the like is utilized when forming the non-linear model.

At 304, one or more processors determine an interval since the last appointment or measurement to be inputted into a prioritization algorithm for determining a priority score. In an example, the time since the last recorded appointment or measurement is determined by utilizing historical data recorded in a memory during a previous visit. Alternatively, the time since the last appointment or measurement is inputted into the one or more computing devices 102 by a clinician at the time of the appointment or measurement. The time determined or inputted may be in units of hours, days, weeks, or the like.

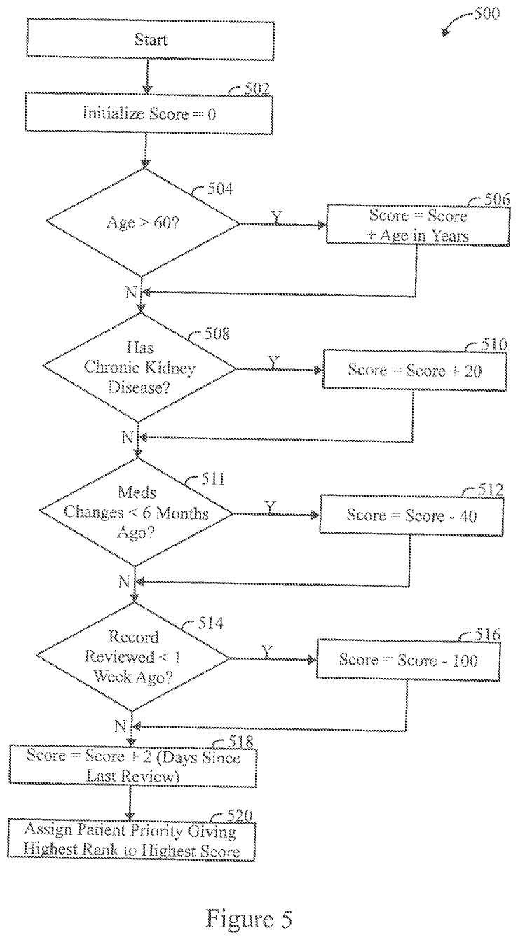

At 306, optionally, the one or more processors determine risk categorization information, including categorizing the risk score with a risk category algorithm. In one example, the risk category algorithm receives risk scores from numerous patients and ranks them in a list and groups the risk scores into categories based on the score. In one example the risk score is provided on a scale from 0-100 with 0 representing a completely healthy individual with no risk of heart failure in the predetermined interval and 100 representing a patient that has a 100% probability of having heart failure in the predetermined interval. In this example, the risk category algorithm assigns any patient having a risk score between 90-100 as highest risk, any patient having a risk score between 80-90 as higher risk, any patient having a risk score between 70-80 as average risk, any patient having a risk score between 60-70 as low risk, and any patient having a risk score less than 60 has minimal risk. In yet another example the risk category algorithm utilizes a color-coded bar with an indicia pointer as illustrated in FIG. 6. In this example, colors may range from a color such as red, representing the highest risk to blue, representing the lowest risk. The indicia pointer then indicates where on the color-coded bar a particular risk score is positioned. In yet another example, the risk category algorithm utilizes a highlighting function to highlight the risk score of any patient with a risk score exceeding a threshold value. This highlight can include bolding the risk score, providing a background color, placing a symbol such as an asterisk by a name or risk score, and the like. In each example the risk category algorithm organizes or categorizes a risk score for use after a risk score of a patient is determined.

At 308, based on the inputs from 302, 304, and 306 a priority patient ranking is determined. In one example, a prioritization algorithm is utilized to make the ranking determinations based on each input. In another example, only one or two of the inputs 302, 304, and/or 306 are utilized. Specifically, the categorizing at 306 in an example may just be utilized as a tool to provide visual information to a clinician. In an example, the prioritization algorithm considers each of the inputs from 302, 304, and 306 as variables and weights are assigned to each input in determining the priority ranking. In one example, the ranking algorithm is an artificial intelligence, or machine learning type algorithm that continuously receives heart failure data including when a patient in a group of patients experiences a heart failure event during a predetermined interval before an appointment is scheduled. The time interval and event are utilized in addition to time intervals of patients that did not experience a heart failure event and had an appointment. Based on these intervals, the weights provided to each of 302, 304, and 306 may be altered.

In yet another example, at 308, ranking is determined by determining patients that entered into a high risk category since their last visit and providing greater or increased weight when determining the rankings to those patients, in one example additional points are added to a determined risk score and a priority score is determined and ranked accordingly. In another example a multiplier is provided to a risk score to determine the priority score and final patient ranking. Alternatively, all patients that have entered into the high risk category are placed in a first category or group and ranked based on risk score to determine prioritization and then all other patients, whether hi the high risk category or otherwise are placed in a lower priority category or group than the group being ranked based on risk score. In this manner, an individual with a lower risk score, but having just entered into a high risk category receives a higher ranking than a patient with a higher risk score, but has been in the high risk category for a predetermined interval of time, such as at a previous appointment that occurred a week before, two weeks before, a month before, and the like.

At 310, the one or more processors determine the final patient ranking. In an example, once the priority patient ranking at 308 is finalized, each patient is provided with an individual ranking. In one example, a methodology as described in relation to 308 is utilized to determine the final patient ranking. Once the final patient ranking is determined, the one or more processors may schedule appointments based on the final patient rankings. In one example the one or more processors send electronic notifications with open dates provided to patients based on their patient ranking, category placement, and the like. The open dates are determined based on such patient ranking, category placement, and the like. Alternatively, a clinician or scheduling personal utilizes the list in scheduling. In one example, medical data related to the patient is inputted into a prioritization algorithm during an appointment as measurement are being detected and determined. This includes inputted directly and automatically from a measurement device or monitor, or from a clinician utilizing measurement and monitoring devices. Based on these inputs the prioritization algorithm determines the patient specific priority rank prior to the appointment terminating, such that a clinician, or scheduling personnel, can coordinate with the patient at the end of the visit to set a follow up visit based on the data received during the appointment or visit. Therefore, higher risk patients have a quicker turn-around and follow-up, reducing risk for a heart failure event, potentially prolonging the patient's life, and saving costs associated therewith.

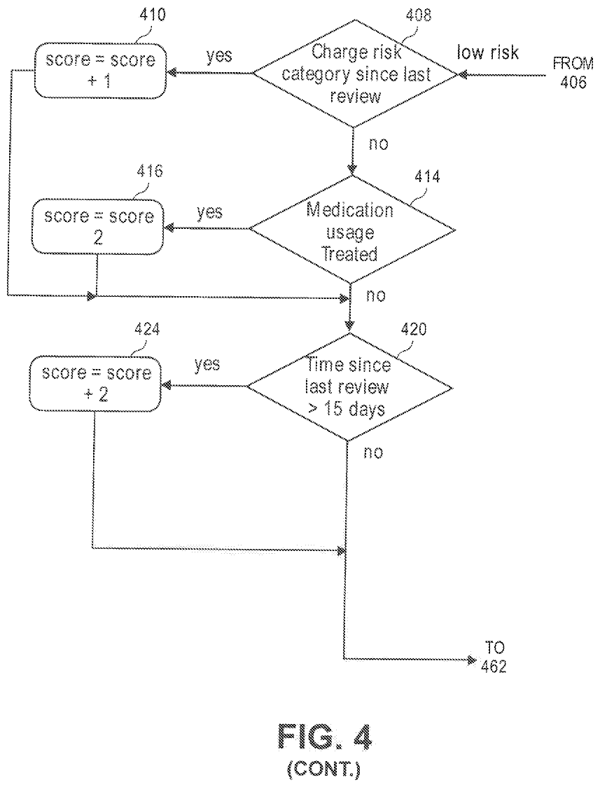

FIG. 4 illustrates a block diagram of yet another method 400 utilized by a clinician management system to determine patient-specific priority rank. In one example the clinician management system is the clinician management system 100 of FIG. 1. In one example this method 400 is performed by the risk score circuitry 124 and prioritization circuitry 126 of FIG. 1. In another example the method 400 is performed by software, including of one or more processors 104 of the one or more computing devices 102, the one or more processors 116 of the monitoring device 114, in the cloud, and the like. In one example, the method 400 is implemented through a patient prioritization algorithm with each determination made through a function or separate algorithm that provides variables for use by the prioritization algorithm.

At 402, an initial score is inputted into a clinician management system. In one example the clinician management system is the clinician management system 100 of FIG. 1. In another example the initial score is inputted into a prioritization algorithm of a clinician management system. Optionally, the risk score is inputted from historical data in a memory. Alternatively and additionally, the initial score is inputted into a computing device through an input device. Also, optionally, the initial score is a default setting of software of the clinician management system. In one example the initial score is set to zero (0) for all patients. Alternatively, a different risk score is utilized such as 10, 100, and the like, with all patients starting with the same initial score. In another example the initial score for patients may be varied based on historical data, as determined by a clinician, or the like.

At 404, one or more processors determine a risk score. In one example the one or more processors utilize a risk score algorithm to determine the risk score. In an example the risk score algorithm determines a probability of a predetermined event and assigns a risk score according. In one example the predetermined event is a heart failure within a predetermined interval of time. The predetermined interval may be one day, one week, two weeks, one month, and the like. In one example, numerous variables are utilized by the risk score algorithm in determining the probability a patient will experience heart failure within the predetermined interval of time. The variables include historical data, pulmonary artery pressure, heart rate, medication usage, and the like. The historical data can include previous patient risk estimates, patient co-morbidities, demographics, medication changes, and the like. Based on these variables the risk score algorithm determines the probability of heart failure within the predetermined interval of time in order to determine the risk score.

In another example, the risk score is determined by the one or more processors by forming a linear model. In an example, the linear model is formed by receiving systolic PAP, diastolic PAP, and heart rate data from an existing group of patients and creating a generalizable estimator based on the received systolic PAP, diastolic PAP, and heart rate data from the existing group of patients. In one example the generalizable estimator utilizes measured pulmonary artery pressure to form the linear model. In yet another example, the risk score is determined by forming a non-linear model. In one example, pulmonary artery pressure data, systolic PAP, diastolic PAP, historical data, and the like is utilized when forming the non-linear model.

At 406, the one or more processors categorize the risk score with a risk category algorithm. In one example, the risk category algorithm receives risk scores from numerous patients and ranks them in a list and groups the risk scores into categories based on the score. In one example the risk score is provided on a scale from 0-100 with 0 representing a completely healthy individual with no risk of HF in the predetermined interval and 100 representing a patient that has a 100% probability of having HF in the predetermined interval. Optionally, a clinician determines the amount of weigh different factors or variables such as the risk score, change in risk score, medication usage titration, time since a previous appointment, visit, or measurement, and the like. The clinician can then also determine the amount of points in a risk score and what ranges of scores are considered a high risk, medium list, low risk, minimal risk, or the like.

In this example, the risk category algorithm assigns any patient having a risk score between 90-100 as highest risk, any patient having a risk score between 80-90 as higher risk, any patient having a risk score between 70-80 as average risk, any patient having a risk score between 60-70 as low risk, and any patient having a risk score less than 60 minimal risk. In yet another example the risk category algorithm utilizes a color-coded bar with an indicia pointer as illustrated in FIG. 6. In this example, colors may range from a color such as red, representing the highest risk to blue, representing the lowest risk. The indicia pointer then indicates where on the color-coded bar a particular risk score is positioned. In yet another example, the risk category algorithm utilizes a highlighting function to highlight the risk score of any patient with a risk score exceeding a threshold value. This highlight can include bolding the risk score, providing a background color, placing a symbol such as an asterisk by a name or risk score, and the like. In each example the risk category algorithm organizes or categorizes a risk score for use after a risk score of a patient is determined. In the example method 400, the one or more processors categorize the risk scores into three categories, low risk, medium risk, and high risk. Alternatively, additional or less risk categories may be determined and utilized.

At 406, flow goes to the left as a result of a low risk being determined at 406, and at 408 a determination is made regarding whether a change in risk category has occurred since a last appointment, measurement, or review. In one example, the risk score is based on a scale from 0-100 where a score between 0-60 is a minimal risk and a score from 61-70 is a low risk score. Therefore, in one example, historical data is provided that includes a risk score of a patient from a previous visit. Alternatively, the historical data includes a median score risk score from multiple previous visits. In yet another example the historical data includes an average risk score from multiple previous visits. Additionally and alternatively, the historic data includes a risk score calculated by taking a weighted average of previous risk scores with more recent test scores receiving greater weight. Optionally, the median, average, weighted average, and the like may be calculated and stored as historical data, or the multiple previous risk scores may be provided from the historical data and the one or more processors may calculate the median, average, weighted average, and the like. Additionally, the risk score utilized may be a risk score from a previous visit or visits, or a priority score from a previous visit or visits.

At 408, when a change in risk category has occurred, then at 410 points are added to the score. In one example, fifteen points are added (+15) to the score. In one example, the risk score of a patient from a previous visit was 55, falling below 60 and thus presenting minimal risk. At the current appointment, measurements are taken, and the risk score is provided as 65, thus placing the patient in the low risk category. As a result, the fifteen points are added, resulting in an overall score of 15.

At 408, when a change in risk category has not occurred, then no points are added, and a determination is made at 414 whether medication usage has been titrated. Titration is the alteration of a dose of medicine in an attempt to prevent a predetermined event such as heart failure in a predetermined time interval. In an example, a clinician inputs whether a medication dosage has been increased or decreased in an attempt to treat the medical condition causing the heart failure. Because of this change in treatment, the probability of a heart failure during a predetermined period should decrease if the alteration in medication is successful. Consequently, if medication usage has been titrated during an appointment or visit, at 416 the risk score is decreased by a predetermined amount. In the example of FIG. 4, the risk score decreases or is subtracted by thirty (−30) points. Therefore, in an example where a risk category has changed, but titration, or medication level change, has been used to combat the increased risk, the score is at negative fifteen (−15) points.

Alternatively, if medication usage is not titrated at 414, then the risk score is not changed, and a determination is made at 420 whether a predetermined interval has occurred since a last visit, appointment, or measurement. In one example, the predetermined interval includes one week, two weeks, one month, and the like. If the predetermine interval has not been exceeded, then no additional points are added to the risk score. If at 420 a predetermined interval has been exceeded, then at 424 additional points are added. In one example, at 424, a scale is provided and the longer a patient has gone without an appointment the more points that are added to the risk score. In one example, when the amount of days from a previous appointment is between 0-7 days, no points are provided, if between 7 and 14 days 20 points are provided, if between 14 and 21 days 30 pts are provided, if between 21 and 28 days 50 pts are added. Thus, points increase to ensure patients are not prioritized out by the patient prioritization algorithm causing them not to be seen indefinitely. Once these determinations are made, a final score is estimated by the patient prioritization algorithm.

At 406, if flow goes to the middle as a result of a medium risk being determined at 406, at 426 a determination is made regarding whether a change in risk category has occurred since a last appointment, measurement, or review. In one example, the risk score is based on a scale from 0-100 where a score between 0-60 is a minimal risk and a score from 61-70 is a low risk score. Therefore, in one example, historical data is provided that includes a risk score of a patient from a previous visit. Alternatively, the historical data includes a median score risk score from multiple previous visits. In yet another example the historical data includes an average risk score from multiple previous visits. Additionally and alternatively, the historic data includes a risk score calculated by taking a weighted average of previous risk scores with more recent test scores receiving greater weight. Optionally, the median, average, weighted average, and the like may be calculated and stored as historical data, or the multiple previous risk scores may be provided from the historical data and the one or more processors may calculate the median, average, weighted average, and the like. Additionally, the risk score utilized may be a risk score from a previous visit or visits, or a priority score from a previous visit or visits.

At 426, when a change in risk category has occurred, then at 428 thirty points are added (+30) to the score. In one example, the risk score of a patient from a previous visit was 65, falling between 60-70 and thus presenting low risk. At the current appointment, measurements are taken, and the risk score is provided as 75, thus placing the patient in the medium risk category. As a result, the thirty points are added, resulting in an overall score of 30.

At 426, when a change in risk category has not occurred, then no points are added, and a determination is made at 432 whether medication usage has been titrated. In an example, a clinician inputs whether a medication dosage has been increased or decrease in an attempt to treat the medical condition causing the heart failure. Because of this change in treatment, the probability of a heart failure during a predetermined period should decrease if the alteration in medication is successful. Consequently, if medication usage has been titrated during an appointment or visit, at 434 the risk score is decreased by a predetermined amount. In the example of FIG. 4, the risk score decreases by thirty (−30) points, or 30 points are subtracted. Therefore, in an example where a risk category has changed, but titration, or medication level change, has been used to combat the increased risk, the score is zero (0) points.

Alternatively, if medication usage is not titrated at 432, then the risk score is not changed, and a determination is made at 438 whether a predetermined interval has occurred since a last visit, appointment, or measurement. In one example, the predetermined interval includes one wee, two weeks, one month, and the like. If the predetermine interval has not been exceeded, then no additional points are added to the risk score. If at 438 a predetermined interval has been exceeded, then at 442 additional points are added. In one example at 442 a scale is provided and the longer a patient has gone without an appointment the more points that are added to the risk score. In one example, when the amount of days from a previous appointment is between 0-4 days, no points are provided, if between 4 and 8 days 20 points are provided, if between 8 and 12 days 30 pts are provided, if between 12 and 16 days 50 pts are added. Thus, points increase to ensure patients are not prioritized out by the patient prioritization algorithm causing them not to be seen indefinitely. Once these determinations are made, a final score is estimated by the patient prioritization algorithm.

At 406, if flow goes to the right as a result of a high risk being determined at 406, at 444 a determination is made regarding whether a change in risk category has occurred since a last appointment, measurement, or review. In one example, the risk score is based on a scale from 0-100 where a score between 0-60 is a minimal risk and a score from 61-70 is a low risk score. Therefore, in one example, historical data is provided that includes a risk score of a patient from a previous visit. Alternatively, the historical data includes a median score risk score from multiple previous visits. In yet another example the historical data includes an average risk score from multiple previous visits. Additionally and alternatively, the historic data includes a risk score calculated by taking a weighted average of previous risk scores with more recent test scores receiving greater weight, Optionally, the median, average, weighted average, and the like may be calculated and stored as historical data, or the multiple previous risk scores may be provided from the historical data and the one or more processors may calculate the median, average, weighted average, and the like. Additionally, the risk score utilized may be a risk score from a previous visit or visits, or a priority score from a previous visit or visits.

At 444, when a change in risk category has occurred, then at 446, seventy-five points are added (+75) to the score. In one example, the risk score of a patient from a previous visit was 65, falling between 60-70 and thus presenting low risk. At the current appointment, measurements are taken, and the risk score is provided as 95, thus placing the patient in the high risk category. As a result, the seventy-five points are added, resulting in an overall score of 75.

At 444, when a change in risk category has not occurred, then no points are added, and a determination is made at 450 whether medication usage has been titrated. In an example, a clinician inputs whether a medication dosage has been increased or decrease hi an attempt to treat the medical condition causing the heart failure. Because of this change in treatment, the probability of a heart failure during a predetermined period should decrease if the alteration in medication is successful. Consequently, if medication usage has been titrated during an appointment or visit, at 452 the risk score is decreased by a predetermined amount. In the example of FIG. 4, the risk score decreases, or is subtracted by thirty (−30) points. Therefore, in an example where a risk category has changed, but titration, or medication level change, has been used to combat the increased risk, the score is forty-five (45) points.

Alternatively, if medication usage is not titrated at 450, then the risk score is not changed, and a determination is made at 456 whether a predetermined interval has occurred since a last visit, appointment, or measurement. In one example, the predetermined interval includes one week, two weeks, one month, and the like. If the predetermine interval has not been exceeded, then no additional points are added to the risk score. if at 456 a predetermined interval has been exceeded, then at 460 additional points are added. In one example, at 460 a scale is provided and the longer a patient has gone without an appointment the more points that are added to the risk score. In one example, when the amount of days from a previous appointment is between 0-2 days, no points are provided, if between 2 and 4 days 20 points are provided, if between 4 and 6 days 30 pts are provided, if between 6 and 8 days 50 pts are added. Thus, points increase more quickly than other legs of the method 400 to ensure higher risk patients are seen more quickly, even when titration has occurred. Once these determinations are made, at 462, a priority score is estimated by the patient prioritization algorithm.

After the priority score is estimated by the patient prioritization algorithm of FIG. 4, priority ranking for each patient is assigned starting with the highest score to the lowest score. In the case of a patient score tie, a tie-breaking methodology is utilized. In one example a tie-breaking calculation is performed depending of each risk category using the equation L/c where L is the length of time since the last appointment, visit, or measurement for a patient and c is a constant dependent on the patient's risk category. In an example c is 2 for a high risk, 5 for medium risk, and 15 for low risk. Then patients with the highest tie-breaking result are given the higher ranking. Alternatively, ties may be broken by random choosing of a patient such as through alphabetical order, reverse alphabetical order, coin flip, and the like. In each case, priority is provided in scheduling based on variables such as change in risk score, medicine usage titration, and the amount of time since a previous appointment, visit, or measurement. Consequently, scheduling is improved, patient health risks are more effectively mitigated, and healthcare costs associated with such health risks is significantly reduced.

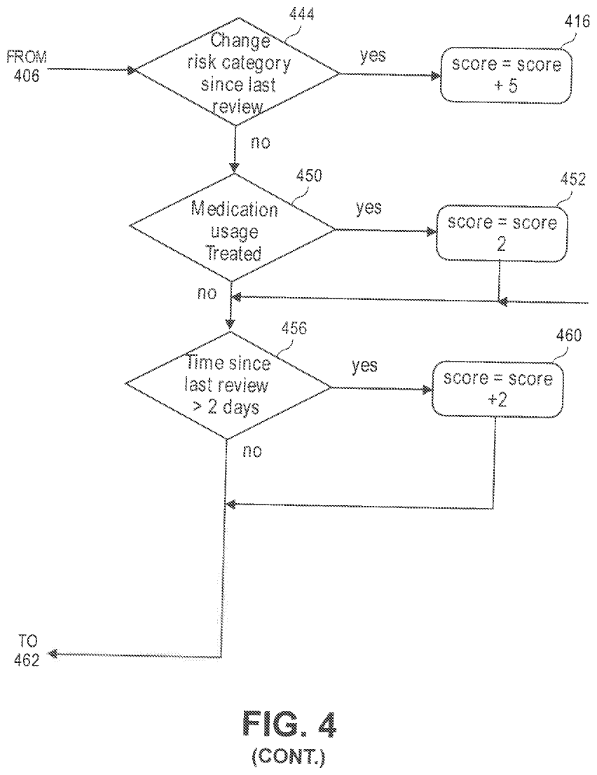

FIG. 5 illustrates a block diagram of another method 500 utilized by a clinician management system to determine patient-specific priority rank. In one example the clinician management system is the clinician management system 100 of FIG. 1. In one example this method 500 is performed by the risk score circuitry 124 and prioritization circuitry 126 of FIG. 1. Alternatively, the patient-specific priority rank does not depend on a separate computation of a risk score. In another example the method 500 is performed by software, including of one or more processors 104 of the one or more computing devices 102, the one or more processors 116 of the monitoring device 114, in the cloud, and the like. In one example, the method 500 is implemented through a patient prioritization algorithm with each determination made through a function or separate algorithm that provides variables for use by the prioritization algorithm.

At 502, an initial score is inputted into a clinician management system. In one example the clinician management system is the clinician management system 100 of FIG. 1. In another example the initial score is inputted into a prioritization algorithm of a clinician management system. Optionally, the initial score is inputted from historical data in a memory. Alternatively and additionally, the initial score is inputted into a computing device through an input device. Also, optionally, the initial score is a default setting of software of the clinician management system. In one example the initial score is set to zero (0) for all patients. Alternatively, a different initial score is utilized such as 10, 100, and the like, with all patients starting with the same initial score. In another example the initial score for patients may be varied based on historical data, as determined by a clinician, or the like.

At 504, a determination is made regarding a first variable. In one example the first variable is the age of the individual. Thus, optionally, if the age of a patient exceeds a threshold age, then at 506 the risk score is increased by the age of the patient. In one example the predetermined age is sixty (60), thus if a patient is 65 and the initial score of the patient is 0, then the new score for the patient is 65. Alternatively, if the patient is 55 years old and the threshold age is 60, then no points are added and at 508 a determination is made regarding a second variable.

At 508, the second variable in one example is a health condition of the patient. Optionally, the health condition relates to whether the patient has kidney disease. In other examples the preexisting health condition could be a previous heart attack, liver disease, heart disease, cancer, and the like. In each case if the patient has the health condition at 510 a predetermined amount of points is added to the score. In one example this could be 20 points such that is a patient is 65 and has kidney disease the patient's updated risk score would be 85.

At 508, if the determination is made that a second variable such as a preexisting health condition doesn't exist, then no points are added to the score and a determination is made at 511 whether medication usage has been titrated at some point during a predetermined period. In an example, a clinician inputs whether a medication dosage has been increased or decreased in an attempt to treat the medical condition causing the heart failure at some point over the past six (6) months. Because of this change in treatment, the probability of a heart failure during a predetermined period should decrease if the alteration in medication is successful. Consequently, if medication usage has been titrated during an appointment or visit, at 512 the risk score is decreased by a predetermined amount. In the example of FIG. 5, the risk score decreases by forty (−40) points. Therefore, in an example where a risk category has changed, but titration, or medication level changes, has been used to combat the increased risk. the score is adjusted accordingly.

Alternatively, if medication usage is not titrated at 511, then the risk score is not changed, and a determination is made at 514 whether a threshold interval has occurred since a last visit, appointment, or measurement. In one example, the predetermined interval includes one week, two weeks, one month, and the like. If the threshold interval has not been exceeded, then at 516 one hundred is subtracted from the score. If at 514 a threshold interval has been exceeded, then at 518 additional points are added, In one example the amount of points added is the amount of days over a threshold amount of day times a multiplier, such as two. Thus, points increase as the days past the threshold interval increase. Once these determinations are made, a priority score is estimated by the patient prioritization algorithm.

After the priority score is estimated by the patient prioritization algorithm of FIG. 5 at 520, priority ranking for each patient is assigned starting with the highest score to the lowest score. In the case of a patient score tie, a tie-breaking methodology can be utilized as discussed in relation to the methodology of FIG. 4. In each case, priority is provided in scheduling based on variables such as change in risk score, medicine usage titration, and the amount of time since a previous appointment, visit, or measurement. Consequently, scheduling is improved, patient health risks are more effectively mitigated, and healthcare costs associated with such health risks is significantly reduced.

FIGS. 3-5 provide different methodologies related to patient prioritization algorithms that may he used to assign a priority based rank for each patient to assist in scheduling patients accordingly. In each of the methods of FIGS. 3 and 4 a risk estimator makes a risk score determination or estimation while the patient prioritization algorithms of FIGS. 3 and 4 use this determination as an input to ultimately assign the rank of each patient.

In one example the risk estimator is an algorithm that uses PAP data from a patient to estimate risk of HF related hospitalization in predetermined interval that in one example is an amount of days at any given point in time. Specifically, the following basic features are extracted from PAP waveforms: systolic PAP data (PAP_{systolic}), diastolic PAP data (PAP_{diastolic}), average PAP (PAPₘₑₐₙ), heart rate, and the like. Derived features include PAP variance, heart rate variability, respiratory modulations, cardiac output, min dP/dt, max dP/dt, mean dP/dt, dicrotic notch time, dicrotic notch pressure, and the like. Patient demographics are also utilized to extract comorbid disease state features including baseline ejection fraction including <or >40%, presence or absence of chronic obstructive pulmonary disease, coronary artery disease, diabetes mellitus, history of myocardial infarction, hyperlipidemia, hypertension, atrial tachycardia flutter/fibrillation, angiotensin converting enzyme (ACE), angiotensin receptor blockers (ARB), beta blockers, race, gender, body mass index (BMI), creatinine, glomerular filtration rate (GFR), blood urea nitrogen (BUN), cardiac resynchronization therapy (CRT-D), and peripheral vascular resistance (PVR).

In one example, in order to determine the features that are most relevant for prediction of hospitalization, one or more processors initially assigns a rank to the above-mentioned features according to their clinical importance for hospitalization prediction. A feature matrix is then developed:

$${X}_{n} = \{ \begin{matrix}[ {X}_{n - 1} {x}_{n} ] & \text{if validation performance is improved} \\ {X}_{n - 1} & \text{otherwise}\end{matrix}$$

recursively at each step of the algorithm, where xₙ is the nth ranked feature. Thus, new features may be temporarily added to the model, and are only retained if the risk score algorithm determines the feature, measurement, or variable results in a predictive value. If a new feature, measurement, or variable does not result in a predictive value the feature, measurement, or variable is discarded.

In order to determine if a new feature, measurement, or variable (hereinafter “feature”) results in a predictive value trend information for such feature matrix is incorporated as current spot values (X(t)), average over a current predetermined interval that in one example is a week (X̅(t)), average over a first previous related interval that in an example is previous week (X̅(t−7)), average over a second previous interval that in one example is two weeks before previous week (X̅(t−14)) and baseline values that in one example is the average over first week (X̅(0)) where the single bar over the X represents an average over one week of historical data, two bars over the X represents an average over two weeks of historical data, and t represents the day during the one week period (t−7), or two week period (t−14) when the period is to begin for collecting the historical data. The expanded matrix is then normalized to prevent value-scaling problems as provided below:

Zₙ=zscore([Xₙ(t), X̅ₙ(t), X̅ₙ(t−7), X̅ₙ(t−14), X̅ₙ(t−28), X̅ₙ(0)])

Subsequently, in one example, in order to de-noise the data, the principal component computation is conducted as Zₙ=UₙSₙVₙ′. Computing the Z-score and principal components give normalized and linearly independent features. These are ranked based on their variance. Using this ranking the matrix is then created reflecting the newest feature matrix with unnecessary dimensions removed as provided:

$${\tilde{U}}_{n , m} = \{ \begin{matrix}[ {\tilde{U}}_{n , m - 1} {u}_{n , m} ] & \text{if validation performance is improved} \\ {\tilde{U}}_{n , m - 1} & \text{otherwise}\end{matrix}$$

Here u_{n,m} is the m^{th} left principal vector at each sub-step of the algorithm such that Uₙ=[u_{n,1}, u_{n,2} . . . , u_{n,d}] where d is the rank, Zₙ·u_{n,m} is a vector of length equivalent to the sum of all time-steps recorded for contributing patients, where m goes from 1 to d. Additionally, n captures the feature that is being considered in this iteration of the top-level algorithm while m captures which specific part of the matrix Ũ that is being referenced. The noise reduction applied to Zₙ helps ensure good generalization of our algorithm on test data.

The multivariate modeling is then done, where the outcome variable indicating a hospitalization in predetermined interval, such as the next d days, then in one example Yₜ is regressed with Ũ_{n,m}(t) using following relations:

Yₜ=f(Ũ_{n,m}(t))

Using this methodology in an experiment, logistic regression-based classification using linear features, quadratic features, quadratic discriminant analysis and neural networks was provided. In other example mean square regression modeling is provided. All model performance—whether the best principal component set for a given physiological set, best physiological feature set, or best regression model structure was cross validated using ten (10) Monte Carlo simulations of six (₆)-fold cross validation, where four (4) sets, one (1) for validation and one (1) for the experiment. In the experiment, the above algorithm and tested to determined feasibility using PAP data from patients. In the experiment machine learning algorithms classified patients into risk groups of probability of hospitalization in next seven or thirty days such that d=7 or 30 days.

Based on these assumptions, the logistic regression models with as few as eight 8 variables, when based on diastolic PAP, systolic PAP and heart rate, were able to provide considerable information patients' risk of a hypervolemic hospitalization in next 7 or 30 days. Particularly, patients were classified or categorized into three groups containing 20%, 30% and 50% of the population, respectively. In this breakdown, the highest 20% group had about 2.6 times higher risk than the average patient, the middle 30% group was at the same risk, and the lowest group containing 50% of the population was at about 0.36 times risk of an impending hospitalization. Thus, patients in the top 20% group were, on average, approximately seven (7) times more likely to be hospitalized for heart failure as opposed to the average patient from the lowest risk group, or half of the population.

While the experiment validated the algorithm, in other examples the algorithm may be implemented via more complex linear and non-linear models with at least twenty (20) to two-hundred and twenty (220) variables, or parameters, wherein the parameters may be based on PA pressure waveform features like dP/dt, breathing modulations, and the like.

By utilizing this risk estimator, clinical monitoring may be streamlined by improving efficiencies at the heart failure clinic. For example, the clinic may use the risk score in a prioritization algorithm, including the prioritization algorithms of FIGS. 3 and 4 to focus on the relevant high risk population rather than reviewing all stable patients as well. In addition, the risk estimator results in a reduction in heart failure hospitalizations and thereby reduce 30-day readmission and the concomitant penalty, therefore reducing healthcare costs on the patient. The risk score algorithm also informs the clinician in regard to a medication prescription. For example, heart failure patients who recently become higher/lower risk may receive a change in medication, such as an increase or decrease in loop diuretics, as compared to patients who continued in their risk group who may not need a change in medication, i.e. the baseline group stratification and also the temporal change in group will be used to make modifications in medication dosages. Initial testing analysis has found that even with very basic linear/logistic regression, a population can be divided into three groups with significantly different risk of an impending hospitalization. Therefore, improved methodologies are provided.

Therefore, by utilizing the risk estimator and accompanying data, a clinician may provide these changes in medication as information and data is being continually updated, including from other patients. Thus, the system is able to eliminate some patients from even needing an appointment in order to change medications and/or medication dosages. Instead such changes may be made remotely, saving both patient and clinician time while improving treatment through implementing such changes in medication and treatment more quickly.

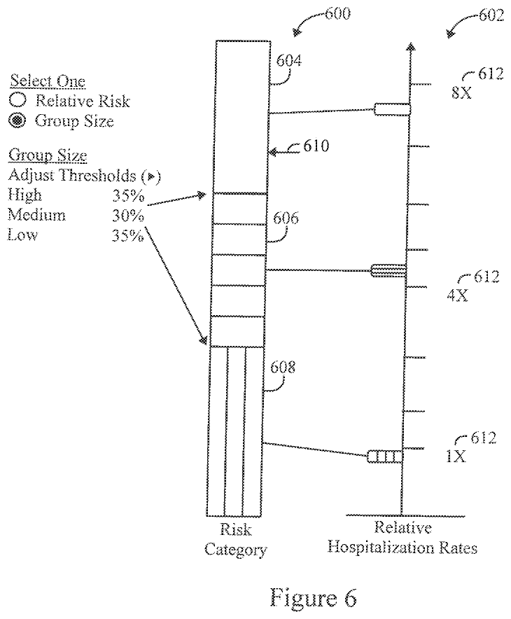

FIGS. 6-7 illustrate example displays the communicate information such as risk score, priority score, risk category, patient ranking, patient ranking indicator, and the like to a clinician. FIG. 6 illustrates a bar graph 600 with an associated measurement indicator 602 used to illustrate and communicate the risk or probability of a predetermined event in a predetermined interval. In one example, the bar graph 600 and measurement indicator represent the probability a patient will be hospitalized for heart failure over the next week. In one example the bar graph 600 is color coded and broken into the sections 604, 606, and 608 with the first section 604 being a first color, the second section 606 being a second color, and the third section 608 being a third color. In one example the first color is red and represents the highest risk group, the second color is yellow representing a medium risk group, and the third section 608 is green, representing a lowest risk group. In this manner a risk score can be matched with a color to immediately indicate to a clinician or scheduler the risk for the patient. In one example an indicia indicator 610 such as an arrow is utilized to point to the exact location on the bar graph 600 a risk score falls to simplify use for a clinician or scheduler.

The measurement indicator 602 meanwhile includes text indicia 612 that includes numbers to provide a numerical value to the risk score. In one example the numbers represent a multiplier with a patient with a score in the eight (8) range being eight times more likely to be hospitalized in the next week for heart failure than an individual with a one (1) score. Additionally, while in one example patients may be categorized based on relative risk, alternatively, they may also be assigned a rank based on group size, utilizing either the bar graph 600 or measurement indicator 602. Thus, information may be presented in such a way to facilitate scheduling for a clinician or scheduler, thus saving time, improving efficiencies, and saving cost.

FIG. 7 illustrates an example display screen 700 utilized by the clinician management system. In one example, the display screen 700 is the interface 112 of clinician management system 100 of FIG. 1. In an example, the display screen 700 includes text indicia 702 that indicates the different, variables, parameters, features, measurements, and the like utilized in determining a priority score. hi this manner, a clinician may review the data, or variables, parameters, features, measurements, and the like to help make determinations such as titration of medication, along with scheduling of future appointments, visits, or measurements.

As an example, the text indicia may include the patient's name, status of change in risk category group, time since last appointment, visit, or measurement, and the variables, parameters, features, measurements, and the like that are responsible for increasing the patient's risk score. These variables, parameters, features, measurements, and the like, may include heart rate, pulse pressure, and the like. While a list of all variables used in the algorithm can be illustrate as part of indicia and graphs displayed, optionally, the one or more processor may selectively display these variables such that only variables that have significantly contributed to the risk score or prioritization score for a particular patient are visualized. Specifically, variables included in the algorithm that did not meaningfully raise the risk level, or score, for a given patient may be excluded, allowing healthcare providers to focus on important factors, facilitating record review. The one or more processors may also provide in the text indicia the chronic or historic factors that are causing a higher risk score, while also classifying new factors that are causing the increase in risk score.

Consequently, by utilizing these visualization tools, the clinician may identify why the risk score has increased, facilitating decision making regarding titration of medication, scheduling, and the like. Thus, by presenting the information in this manner the clinician management system may be utilized as a tool to inform medication and diagnosis decisions, improving patient care in addition to improving patient scheduling.

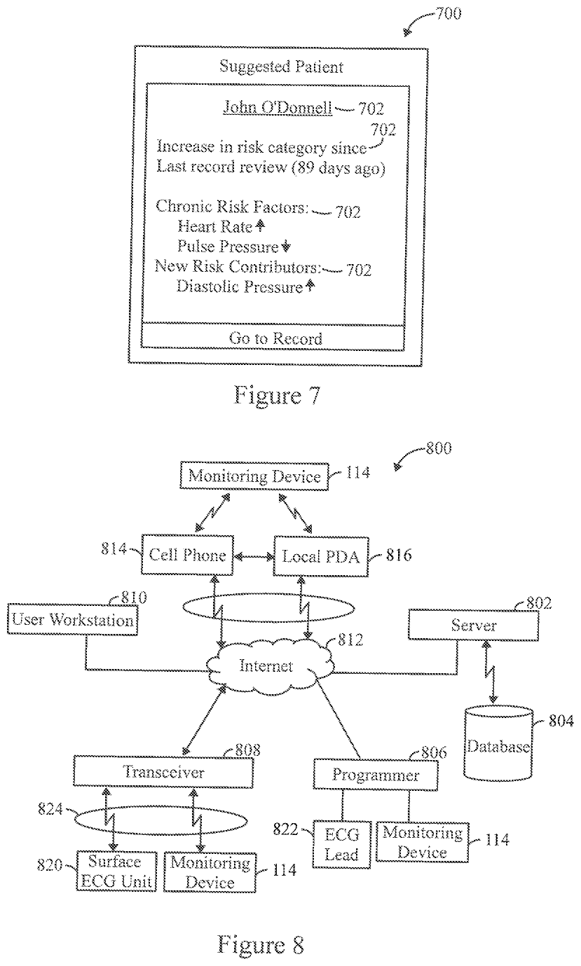

FIG. 8 illustrates a distributed processing system 800 in accordance with one embodiment. The distributed processing system 800 includes a server 802 connected to a database 804, a programmer 806, a local RF transceiver 808 and a user workstation 810 electrically connected to a communication system 812. Any of the processor-based components in FIG. 8 (e.g., workstation 810, cell phone 814, PDA 816, server 802, programmer 806, monitoring device 114) may perform the COI measurement process discussed above.

The communication system 812 may be the internet, a voice over IP (VoIP) gateway, a local plain old telephone service (POTS) such as a public switched telephone network (PSTN), a cellular phone based network, and the like. Alternatively, the communication system 812 may be a local area network (LAN), a campus area network (CAN), a metropolitan area network (MAN), or a wide area network (WAM). The communication system 812 serves to provide a network that facilitates the transfer/receipt of information such as cardiac signal waveforms, ventricular and atrial heart rates.

The server 802 is a computer system that provides services to other computing systems over a computer network. The server 802 controls the communication of information such as cardiac signal waveforms, ventricular and atrial heart rates, and detection thresholds. The server 802 interfaces with the communication system 812 to transfer information between the programmer 806, the local RF transceiver 808, the user workstation 810 as well as a cell phone 814 and a personal data assistant (PDA) 816 to the database 804 for storage/retrieval of records of information. On the other hand, the server 802 may upload raw cardiac signals from an implanted lead 822, surface ECG unit 820 or the monitoring device 114 via the local RF transceiver 808 or the programmer 806.

The database 804 stores information such as cardiac signal waveforms, ventricular and atrial heart rates. thresholds, and the like, for a single or multiple patients. The information is downloaded into the database 804 via the server 802 or, alternatively, the information is uploaded to the server from the database 804. The programmer 806 is similar to an external device such as one or more computing devices 102 and may reside in a patient's home, a hospital, or a physician's office. The programmer 806 interfaces with the lead 822 and the monitoring device 114. The programmer 806 may wirelessly communicate with the monitoring device 114 and utilize protocols, such as Bluetooth, GSM, infrared wireless LANs, HIPERLAN, 3G, satellite, as well as circuit and packet data protocols, and the like. Alternatively, a hard-wired connection may be used to connect the programmer 806 to the monitoring device 114. The programmer 806 is able to acquire cardiac signals from the surface of a person (e.g., ECGs), intra-cardiac electrogram (e.g., IEGM) signals from the monitoring device 114, and/or cardiac signal waveforms, ventricular and atrial heart rates, and detection thresholds from the monitoring device 114. The programmer 806 interfaces with the communication system 812, either via the internet or via POTS, to upload the information acquired from the surface ECG unit 820, the lead 822 or the monitoring device 114 to the server 802.

The local RF transceiver 808 interfaces with the communication system 812 to upload one or more of cardiac signal waveforms, ventricular and atrial heart rates, and detection thresholds to the server 802. In one embodiment, the surface ECG unit 820 and the monitoring device 114 have a bi-directional connection 824 with the local RF transceiver 808 via a wireless connection. The local RF transceiver 808 is able to acquire cardiac signals from the surface of a person, intra-cardiac electrogram signals from the monitoring device 114, and/or cardiac signal waveforms, ventricular and atrial heart rates, and detection thresholds from the monitoring device 114. On the other hand, the local RF transceiver 808 may download stored cardiac signal waveforms, ventricular and atrial heart rates, and detection thresholds, and the like, from the database 804 to the surface ECG unit 820, one or more computing devices 102, or the monitoring device 114.

The user workstation 810 may interface with the communication system 812 via the internet or POTS to download cardiac signal waveforms, ventricular and atrial heart rates, and detection thresholds via the server 802 from the database 804. Alternatively, the user workstation 810 may download raw data from the surface ECG units 820, lead 822, or monitoring device 114 via either the programmer 806 or the local RF transceiver 808. Once the user workstation 810 has downloaded the cardiac signal waveforms, ventricular and atrial heart rates, or detection thresholds, the user workstation 810 may process the information in accordance with one or more of the operations described above. The user workstation 810 may download the information and notifications to the cell phone 814, the PDA 816, the local RF transceiver 808, the programmer 806, or to the server 802 to be stored on the database 804. For example, the user workstation 810 may communicate data to the cell phone 814 or PDA 816 via a wireless communication link.

Thus, provided is a clinician management system that determines a priority score of a predetermined event over a predetermined period for each patient. Based on the risk score, each patient is ranked, and future appointments are scheduled to ensure those patients with the greatest risk of a medical emergency are monitored more closely than those with less of a risk. In this manner, unneeded and undesired hospitalizations may be avoided through preventative detection, reducing costs associated with emergency medical procedures. Additionally, such a system also assists in prolonging a human's life, and increases patient care. Thus, and improved system and methodology are provided.

### Closing Statement

It should be clearly understood that the various arrangements and processes broadly described and illustrated with respect to the Figures, and/or one or more individual components or elements of such arrangements and/or one or more process operations associated of such processes, can be employed independently from or together with one or more other components, elements and/or process operations described and illustrated herein. Accordingly, while various arrangements and processes are broadly contemplated, described and illustrated herein, it should be understood that they are provided merely in illustrative and non-restrictive fashion, and furthermore can be regarded as but mere examples of possible working environments in which one or more arrangements or processes may function or operate.

As will be appreciated by one skilled in the art, various aspects may be embodied as a system, method or computer (device) program product. Accordingly, aspects may take the form of an entirely hardware embodiment or an embodiment including hardware and software that may all generally be referred to herein as a “circuit,” “module” or “system.” Furthermore, aspects may take the form of a computer (device) program product embodied in one or more computer (device) readable storage medium(s) having computer (device) readable program code embodied thereon.

Any combination of one or more non-signal computer (device) readable medium(s) may be utilized. The non-signal medium may be a storage medium. A storage medium may be, for example, an electronic, magnetic, optical, electromagnetic, infrared, or semiconductor system, apparatus, or device, or any suitable combination of the foregoing. More specific examples of a storage medium would include the following: a portable computer diskette, a hard disk, a random access memory (RAM), a dynamic random access memory (DRAM), a read-only memory (ROM), an erasable programmable read-only memory (EPROM or Flash memory), a portable compact disc read-only memory (CD-ROM), an optical storage device, a magnetic storage device, or any suitable combination of the foregoing.

Program code for carrying out operations may be written in any combination of one or more programming languages. The program code may execute entirely on a single device, partly on a single device, as a stand-alone software package, partly on single device and partly on another device, or entirely on the other device. In some cases, the devices may be connected through any type of network, including a local area network (LAN) or a wide area network (WAN), or the connection may be made through other devices (for example, through the Internet using an Internet Service Provider) or through a hard wire connection, such as over a USB connection. For example, a server having a first processor, a network interface, and a storage device for storing code may store the program code for carrying out the operations and provide this code through its network interface via a network to a second device having a second processor for execution of the code on the second device.

Aspects are described herein with reference to the figures, which illustrate example methods, devices and program products according to various example embodiments. These program instructions may be provided to a processor of a general purpose computer, special purpose computer, or other programmable data processing device or information handling device to produce a machine, such that the instructions, which execute via a processor of the device implement the functions/acts specified. The program instructions may also be stored in a device readable medium that can direct a device to function in a particular manner, such that the instructions stored in the device readable medium produce an article of manufacture including instructions which implement the function/act specified. The program instructions may also be loaded onto a device to cause a series of operational steps to be performed on the device to produce a device implemented process such that the instructions which execute on the device provide processes for implementing the functions/acts specified.

The units/modules/applications herein may include any processor-based or microprocessor-based system including systems using microcontrollers, reduced instruction set computers (RISC), application specific integrated circuits (ASICs), field-programmable gate arrays (FPGAs), logic circuits, and any other circuit or processor capable of executing the functions described herein. Additionally or alternatively, the modules/controllers herein may represent circuit modules that may be implemented as hardware with associated instructions (for example, software stored on a tangible and non-transitory computer readable storage medium, such as a computer hard drive, ROM, RAM, or the like) that perform the operations described herein. The above examples are exemplary only, and are thus not intended to limit in any way the definition and/or meaning of the term “controller.” The units/modules/applications herein may execute a set of instructions that are stored in one or more storage elements, in order to process data. The storage elements may also store data or other information as desired or needed. The storage element may be in the form of an information source or a physical memory element within the modules/controllers herein. The set of instructions may include various commands that instruct the modules/applications herein to perform specific operations such as the methods and processes of the various embodiments of the subject matter described herein. The set of instructions may be in the form of a software program. The software may be in various forms such as system software or application software. Further, the software may be in the form of a collection of separate programs or modules, a program module within a larger program or a portion of a program module. The software also may include modular programming in the form of object-oriented programming. The processing of input data by the processing machine may be in response to user commands, or in response to results of previous processing, or in response to a request made by another processing machine.

It is to be understood that the subject matter described herein is not limited in its application to the details of construction and the arrangement of components set forth in the description herein or illustrated in the drawings hereof. The subject matter described herein is capable of other embodiments and of being practiced or of being carried out in various ways. Also, it is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items.

It is to be understood that the above description is intended to be illustrative, and not restrictive. For example, the above-described embodiments (and/or aspects thereof) may be used in combination with each other. In addition, many modifications may be made to adapt a particular situation or material to the teachings herein without departing from its scope. While the dimensions, types of materials and coatings described herein are intended to define various parameters, they are by no means limiting and are illustrative in nature. Many other embodiments will be apparent to those of skill in the art upon reviewing the above description. The scope of the embodiments should, therefore, be determined with reference to the appended claims, along with the full scope of equivalents to which such claims are entitled. In the appended claims, the terms “including” and “in which” are used as the plain-English equivalents of the respective terms “comprising” and “wherein.” Moreover, in the following claims, the terms “first,” “second,” and “third,” etc. are used merely as labels, and are not intended to impose numerical requirements on their objects or order of execution on their acts.

What is claimed is:

1. A system for prioritizing patients for clinician management comprising:
one or more processors configured to execute program instructions to:
determine a risk score for a patient;
alter the risk score based on patient based parameters to determine a priority score of the patient;
compare the priority score of the patient to priority scores of other patients;
assign a rank to the patient based on the comparison of the priority score of the patient to the priority scores of the other patients; and
schedule an appointment for the patient based on the rank.

2. The system of claim 1, wherein determining the priority score for a patient comprises:
estimating a probability of a predetermined event to determine the risk score for the patient;
determining a time period since a last appointment;
receiving medication titration data;
inputting the probability of the predetermined event, the time period since the last appointment, and medication titration data into a patient prioritization algorithm.

3. The system of claim 2, wherein estimating the probability of the predetermined event comprises:
monitoring pulmonary artery pressure with a sensor to detect pulmonary artery pressure data;
recording the pulmonary artery pressure data detected by the sensor; and
calculating a risk estimate based on the recorded pulmonary artery pressure data.

4. The system of claim 3, wherein calculating the risk estimate includes determining one of patient heart rate or medication usage.

5. The system of claim 3, wherein estimating the probability of the predetermined event further comprises:
calculating the probability of the predetermined event based on historical data.

6. The system of claim 6, wherein the historical data includes one of previous patient risk estimates, patient co-morbidities, demographics, or medication changes.

7. The system of claim 2, wherein the predetermined event is heart failure.

8. The system of claim 1, wherein determining the risk score for the patient further comprises:
forming a linear model;
receiving systolic pulmonary artery pressure, diastolic pulmonary artery pressure, and heart rate data from an existing group of patients;
creating a generalizable estimator based on the received systolic pulmonary artery pressure, diastolic pulmonary artery pressure, and heart rate data from an existing group of patients;
monitoring pulmonary artery pressure with a sensor to detect pulmonary artery pressure data; and
utilizing the pulmonary artery pressure in with the generalizable estimator.

9. The system of claim 1, wherein determining the risk score for the patient further comprises:
forming a non-linear model;
monitoring pulmonary artery pressure with a sensor to detect pulmonary artery pressure data; and
utilizing the pulmonary artery pressure data in the non-linear model.

10. A system for prioritizing patients for clinician management comprising:
one or more processors configured to execute program instructions to:
receive a risk score for a patient inputted into the one or more processors at an interface;
determine a first variable related to the patient based on historical data;
alter the risk score by a predetermined amount based on the determined first variable to provide an updated risk score;
determine a second variable related to the patient based on sensor data received by the one or more processors;
alter the updated risk score based on the determined second variable to provide a priority score of the patient;
compare the priority score of the patient to priority scores of other patients;
assign a ranking to the patient based on the comparison of the priority score of the patient to the priority scores of the other patients; and
schedule an appointment for the patient based on the ranking.

11. The system of claim 10, wherein the historical data includes one of previous patient risk estimates, patient co-morbidities, demographics, or medication changes.

12. The system of claim 10, wherein altering the risk score by a predetermined amount includes subtracting from the risk score.

13. A method of prioritizing patients for clinician management comprising:
monitoring pulmonary artery pressure with a sensor to detect pulmonary artery pressure data;
estimating the probability a patient will have a heart failure event during a predetermined period based on the detected pulmonary artery pressure data;
determining a risk score for the patient based on the probability the patient will have the heart failure event during a predetermined period;
altering the risk score based on patient based parameters to determine a priority score of the patient;
comparing the priority score of the patient to priority scores of other patients;
assigning a rank to the patient based on the comparison of the priority score of the patient to the priority scores of the other patients; and
scheduling an appointment for the patient based on the rank.

14. The method of claim 13, wherein determining the priority score for the patient comprises:
determining a time period since a last appointment;
receiving medication titration data;
inputting the probability the patient will have the heart failure event during the predetermined period, the time period since the last appointment, and medication titration data into a patient prioritization algorithm.

15. The method of claim 14, further comprising:
determining a first weight related to the inputted probability the patient will have the heart failure event during the predetermined period;
determining a second weight related to the inputted time period since the last appointment; and
determining a third weight related to the inputted medication titration data.

16. The method of claim 14, wherein the medication titration data includes change in medication usage data.

17. The method of claim 13, wherein estimating the probability the patient will have the heart failure event during the predetermined period based on the detected pulmonary artery pressure data further comprises:
receiving historical data related to the probability the patient will have the heart failure event during the predetermined period; and
comparing the historical data to the detected pulmonary artery pressure data.

18. The method of claim 13, wherein estimating the probability the patient will have the heart failure event during the predetermined period based on the detected pulmonary artery pressure data further comprises:
receiving historical data related to the probability the patient will have the heart failure event during the predetermined period; and
inputting the historical data into a risk score algorithm.

19. The method of claim 18, wherein the historical data includes one of previous patient risk estimates, patient comorbidities, demographics, or medication changes.

20. The method of claim 18, wherein detecting pulmonary artery pressure data includes forming a pulmonary pressure waveform and extracting the detected pulmonary artery pressure data from the detected pulmonary pressure waveform; and
wherein the risk score is determined utilizing the risk score algorithm and the detected pulmonary artery pressure data.
